(19) United States
(12) Patent Application Publication
(10) Pub. No.: US 2016/0128557 A1
(43) Pub. Date: May 12, 2016

(54) APPARATUS FOR REMOVING INFRARED (IR) LIGHT FROM AN OPHTHALMIC ILLUMINATION SYSTEM

(71) Applicant: NOVARTIS AG, BASEL (CH)

(72) Inventors: MICHAEL J. PAPAC, NORTH TUSTIN, CA (US); RONALD T. SMITH, IRVINE, CA (US); GERALD D. BACHER, CARLSBAD, CA (US)

(21) Appl. No.: 14/746,342

(22) Filed: Jun. 22, 2015

Related U.S. Application Data
(60) Provisional application No. 62/075,971, filed on Nov. 6, 2014.

(51) Int. Cl. A61B 3/00; A61B 1/06; A61B 1/12; A61F 9/007

(52) U.S. Cl. CPC A61B 3/0008; A61F 9/00736; A61B 1/0646; A61B 1/128; A61B 2019/5206; A61B 2019/5221; A61B 2217/005

(57) ABSTRACT

An ophthalmic illumination apparatus includes a light source operable to generate a light beam and a filtering mirror configured to generate an infrared (IR) filtered light beam by reflecting at least a portion of wavelengths of the light beam in the visible spectrum while filtering out at least a portion of wavelengths of the light beam in the IR spectrum. The ophthalmic illumination apparatus further includes a collimator configured to receive at least a portion of the IR filtered light beam and generate a collimated light beam and a condenser configured to couple at least a portion of the collimated light beam into an optical fiber.

This application claims the priority of U.S. Provisional Application No. 62/075,971 filed Nov. 6, 2014 which is hereby incorporated herein by reference in its entirety.

### FIELD

The present disclosure relates generally to the field of ophthalmic illumination systems and, more particularly, to removing infrared (IR) light from an ophthalmic illumination system.

### BACKGROUND

Ophthalmic illumination probes are used to provide illumination in ophthalmic surgeries. In particular, an ophthalmic illumination probe may be inserted into an eye to provide illumination inside the eye during an ophthalmic surgery. Typically, the ophthalmic illumination probe is connected to an optical port of an ophthalmic illumination system to receive light from the ophthalmic illumination system. The ophthalmic illumination system may include a light source that produces light and a condenser that couples the light into an optical fiber of the ophthalmic illumination probe. During the assembly of the optical port of the ophthalmic illumination system, the position and tilt of the light beam from the condenser is adjusted until a coupling efficiency of the light beam into the ophthalmic illumination probe connected at the optical port reaches an optimal value. Then, the assembly of the optical port is fixed or immobilized to maintain the coupling position and the coupling efficiency of the light beam into the ophthalmic illumination probe. Nevertheless, various factors may cause the coupling position to move, which may result in a loss of coupling efficiency. Examples of such factors include shock and vibration imparted to the optical port assembly during shipment and setup, thermal-induced expansion, rotation and distortion of opto-mechanical mounts used to direct the light beam, thermal-induced motion of the optical fiber port, or beam motion caused by movement of adjustable reflective elements within the system, such as a rotatable or translatable variable beam splitters. The present disclosure is directed to an illumination system that addresses one or more of the above-described disadvantages of current systems.

### SUMMARY

The present disclosure concerns an ophthalmic illumination system in which light emitted from a light source is filtered in a manner that reduces or eliminates the presence of wavelengths in the IR spectrum. As a result, heating of certain components of the ophthalmic illumination system may be reduced, which may reduce or eliminate heat induced distortion of those components. Reducing such distortion may allow the coupling efficiency between the generated light beam and an attached ophthalmic illumination probe to be maintained. Additionally, the IR light reaching the eye, which may be damaging to the eye, may be reduced or eliminated.

In certain embodiments, an ophthalmic illumination apparatus includes a light source operable to generate a light beam and a filtering mirror configured to generate an infrared (IR) filtered light beam by reflecting at least a portion of wavelengths of the light beam in the visible spectrum while filtering out at least a portion of wavelengths of the light beam in the IR spectrum. The ophthalmic illumination apparatus further includes a collimator configured to receive at least a portion of the IR filtered light beam and generate a collimated light beam and a condenser configured to couple at least a portion of the collimated light beam into an optical fiber.

In certain other embodiments, an ophthalmic surgical system includes an ophthalmic illumination apparatus having a light source operable to generate a light beam, a filtering mirror configured to generate an infrared (IR) filtered light beam by reflecting at least a portion of wavelengths of the light beam in the visible spectrum while filtering out at least a portion of wavelengths of the light beam in the IR spectrum, a collimator configured to receive at least a portion of the IR filtered light beam and generate a collimated light beam, and a condenser configured to focus at least a portion of the collimated light beam. The ophthalmic surgical system further includes a connection port comprising an opening into which the condenser focuses the at least a portion of the collimated light beam. The connection port is configured to receive a detachable fiber connector including an optical fiber connected to an ophthalmic surgical tool, and the at least a portion of the collimated light beam focused by the condenser is transmitted by the optical fiber to the ophthalmic surgical tool.

In certain embodiments, an ophthalmic illumination apparatus includes a light source operable to generate a light beam and a filtering mirror configured to generate an infrared (IR) filtered light beam by reflecting at least a portion of wavelengths of the light beam in the IR spectrum while letting pass through at least a portion of wavelengths of the light beam in the visible spectrum. The ophthalmic illumination apparatus further includes a collimator positioned along the same optical axis as the light source, the collimator configured to receive at least a portion of the IR filtered light beam and generate a collimated light beam, and a condenser configured to couple at least a portion of the collimated light beam into an optical fiber.

### BRIEF DESCRIPTION OF THE DRAWINGS

For a more complete understanding of the present disclosure and the advantages thereof, reference is now made to the following description taken in conjunction with the accompanying drawings in which like reference numerals indicate like features and wherein:

FIG. 1 illustrates an exemplary ophthalmic illumination system, according to certain embodiments of the present disclosure;

FIG. 2 illustrates an implementation of the ophthalmic illumination system of FIG. 1 in an exemplary surgical console, according to certain embodiments of the present disclosure;

FIG. 3 illustrates a plot of radiometric flux versus wavelength in order to show the filtering properties of the exemplary ophthalmic illumination system depicted in FIG. 1; and

FIG. 4 illustrates an alternative exemplary ophthalmic illumination system, according to certain embodiments of the present disclosure.

The skilled person in the art will understand that the drawings, described below, are for illustration purposes only. The drawings are not intended to limit the scope of the applicant's disclosure in any way.

### DETAILED DESCRIPTION

For the purposes of promoting an understanding of the principles of the present disclosure, reference will now be made to the embodiments illustrated in the drawings, and specific language will be used to describe the same. It should nevertheless be understood that no limitation of the scope of the disclosure is intended. Any alterations and further modifications to the described systems, devices, and methods, and any further application of the principles of the present disclosure are fully contemplated as would normally occur to one skilled in the art to which the disclosure relates. In particular, it is fully contemplated that the systems, devices, and/or methods described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

In general, the present disclosure may provide an ophthalmic illumination system in which light emitted from a light source is filtered in a manner that reduces or eliminates the presence of wavelengths in the IR spectrum. As a result, heating of certain components of the ophthalmic illumination system may be reduced, which may reduce or eliminate heat induced distortion of those components. Reducing such distortion may allow the coupling efficiency between the generated light beam and an attached ophthalmic illumination probe to be maintained.

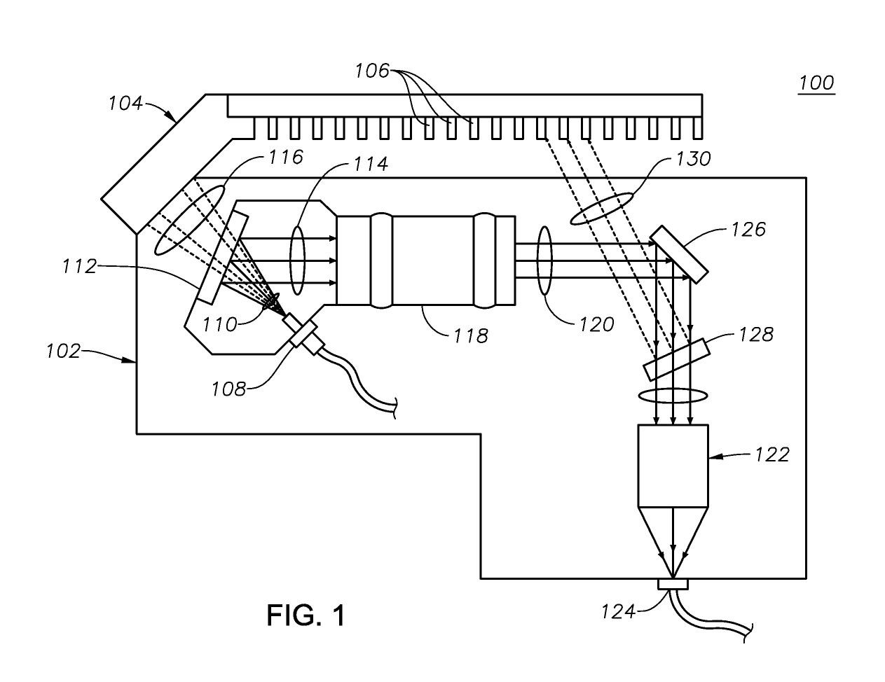

FIG. 1 illustrates an exemplary ophthalmic illumination system 100, according to certain embodiments of the present disclosure. Ophthalmic illumination system 100 may include a base plate 102 to which one or more optical components of the system (as discussed in detail below) are mounted or otherwise fixed. Base plate 102 may be constructed of any suitable material or combination of materials and include any suitable structure or combination of structures facilitating the mounting of the various components of ophthalmic illumination system 100 (described below) in a manner that maintains a desired optical alignment between those components.

Ophthalmic illumination system 100 may further include a heat sink 104 for dissipating heat associated with infrared (IR) light filtered from the light beam 110 generated by light source 108 (as discussed in further detail below). Heat sink 104 may be constructed of any suitable material or combination of materials and may include any suitable structure for dissipating a desired amount of heat. For example, heat sink 104 may be constructed of a highly conductive metallic material that is absorptive of IR light. In certain embodiments, the absorptive region of base plate 104 (i.e., the region upon which IR portion 116 is incident) may be configured to reduce reflection of IR light toward light source 108. For example, absorptive region of base plate 104 may be coated with an antireflective material, such as a dielectric antireflective coating or antireflective nanostructures. As another example, the absorptive region of base plate 104 may be constructed of objects having high aspect ratios to minimize reflection of IR light toward light source 108 (e.g., cones, pyramids, or razor blade type structures).

In certain embodiments, heat sink 104 may include a number of fins 106 that increase the overall surface area of the heat sink 104 such that the heat dissipation capacity of the heat sink is increased. Additionally, heat sink 104 may be thermally isolated from base plate 102 such that any increase in temperature of heat sink 102 does not affect base plate 102 and/or distort components mounted thereto.

Although base plate 102 and heat sink 104 are depicted as having particular shapes/structures and as being oriented relative to one another in a particular manner, the present disclosure contemplates that base plate 102 and heat sink 104 may have any suitable shapes/structures and may be oriented relative to one another in any suitable manner, according to particular needs and consistent with the functionality described herein.

Ophthalmic illumination system 100 may further include a light source 108 operable to generate a light beam 110. In certain embodiments, light source 108 may include may be a supercontinuum source, which may be desirable due to the ability to supply high intensity visible light to ophthalmic illumination system 100. However, because a supercontinuum source may operate by spectrally broadening a supplied pump beam and that spectral broadening may be substantially or at least partially symmetric about the wavelength of the pump beam (e.g., 1064 nanometers), the generation of desirable visible light (e.g., light having wavelengths in the range of 440-650 nanometers) may be accompanied by the generation of IR light (e.g., light having wavelengths greater than 700 nanometers). Because IR light may not provide any functional benefit with regard to ophthalmic illumination and cause undesirable heat generation, it may be desirable to filter out all or a portion of the IR light in the light beam 110 prior to condensing the light beam 110 into a consumable optical fiber facilitating application to the eye of a patient. Although light source 108 is primarily described as being a supercontinuum source, the present disclosure contemplates that light source 108 may include any suitable light source for producing a light beam 110 that may be condensed into a consumable optical fiber, as discussed below.

To facilitate the above-discussed filtering of IR light from light beam 110, ophthalmic illumination system 100 may include filtering mirror 112 disposed in the path of light beam 110. In certain embodiments, filtering mirror 112 may be a dichroic cold mirror coated such that all or a portion of light in the visible spectrum (e.g., IR filtered beam 114) is reflected while all or a portion of light in the IR spectrum (IR portion 116) passes though. Moreover, filtering mirror 112 may be oriented relative to light source 108 such that the reflected visible light (IR filtered beam 114) is directed towards subsequent optics in the ophthalmic illumination system 100 while the IR portion 116 passes through and is incident upon all or a portion of heat sink 104. Consequently, heat associated with the IR portion 116 of light beam 110 may be absorbed and dissipated by heat sink 104, reducing or eliminating heating of (and possible distortion of) subsequent optics in the ophthalmic illumination system 100.

Although filtering mirror 112 is depicted and described as being a flat dichroic cold mirror positioned at an angle relative to light source 108, the present disclosure contemplates that filtering mirror 112 may include any suitable mirror (e.g., convex, concave, etc.) operable to pass IR light while reflecting visible light to any suitable any suitable location.

The IR filtered beam 114 reflected by filtering mirror 112 may be received may a collimator 118 comprising optics operable to generate a collimated light beam 120. The present disclosure contemplates that collimator 118 may comprise any suitable optic or combination of optics operable to produce a collimated light beam 120. Because all or a portion of the IR light (IR portion 116) was filtered from light beam 110 by filtering mirror 112, the amount of IR light absorbed by the housing of collimator 118 may be reduced or eliminated. Moreover, because the housing of collimator 118 may be susceptible to thermal expansion, reducing or eliminating the amount of IR light absorbed by the housing of collimator 118 may advantageously reduce or eliminate such thermal expansion. As a result, distortion of the optics of collimator 118 may be reduced or eliminated such that proper alignment between the components of ophthalmic illumination system 100 may be maintained (which may be increasingly important as the diameter of consumable optical fiber decreases).

The collimated light beam 120 generated by collimator 118 may be directed to a condenser 122 configured to couple the collimated light beam 120 into a consumable optical fiber 124. The condenser 122 may include one or more optical lenses or other optical components configured to focus the collimated light beam 120 into a consumable optical fiber of desirable size. For example, condenser 122 may be configured such that collimated light beam 120 is focused in a manner suitable for coupling to a consumable optical fiber 122 having a 25 μm core. However, optical fibers having any suitable diameters or sizes may be used.

In certain embodiments, the orientation of collimator 118 relative to condenser 122 may necessitate that collimated light beam 120 be redirected after exiting collimator 118. For example, ophthalmic illumination system 100 may include a fold mirror 126 configured to reflect collimated light beam 120 toward condenser 124.

In certain embodiments, it may be desirable to further filter collimated light beam 118 of IR light prior to focusing collimating light beam via condenser 122. For example, in instances where the coating of filtering mirror 112 (e.g., a dichroic cold mirror) is not perfect, a small percentage of IR light may remain in filtered light beam 114 and, as a result, collimated light beam 120. To avoid possible heat related distortion of condenser 122 and/or IR light entering consumable optical fiber 124, it may be desirable to include an additional filtering mirror 128. For example, additional filtering mirror 128 may include a hot mirror coated such that such that all or a portion of the remaining IR light of collimated light beam 120 (residual IR light 130) is reflected while the visible portion of collimated light beam 120 passes through to condenser 122. Moreover, additional filtering mirror 128 may be oriented such that the reflected residual IR light 130 is directed towards heat sink 104. Consequently, heat associated with the residual IR light 130 may be absorbed and dissipated by heat sink 104.

Although the above-discussed components of ophthalmic illumination system 100 are depicted and described as having relative positions within the system, the present disclosure contemplates that the various components of system 100 may have any suitable position relative or absolute positioning, according to particular needs.

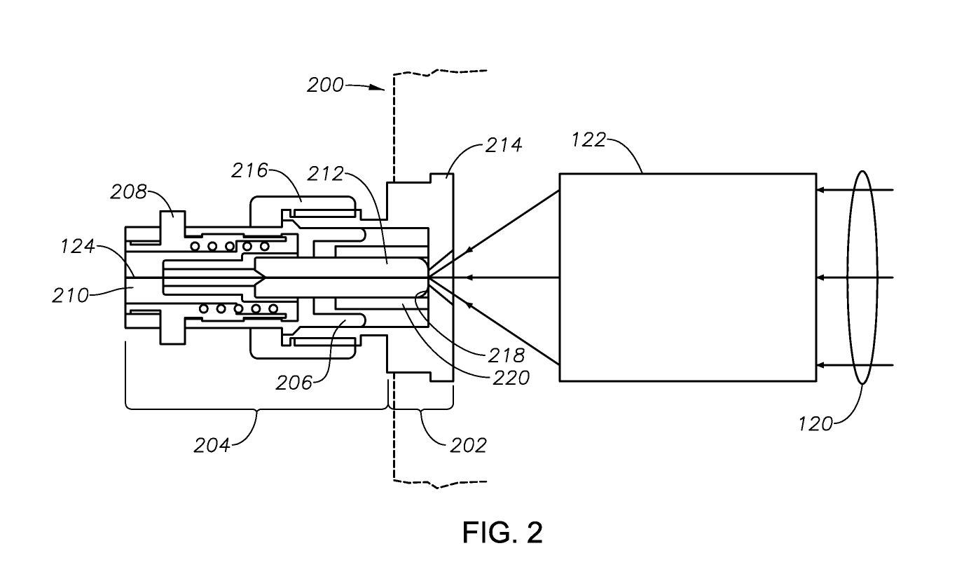

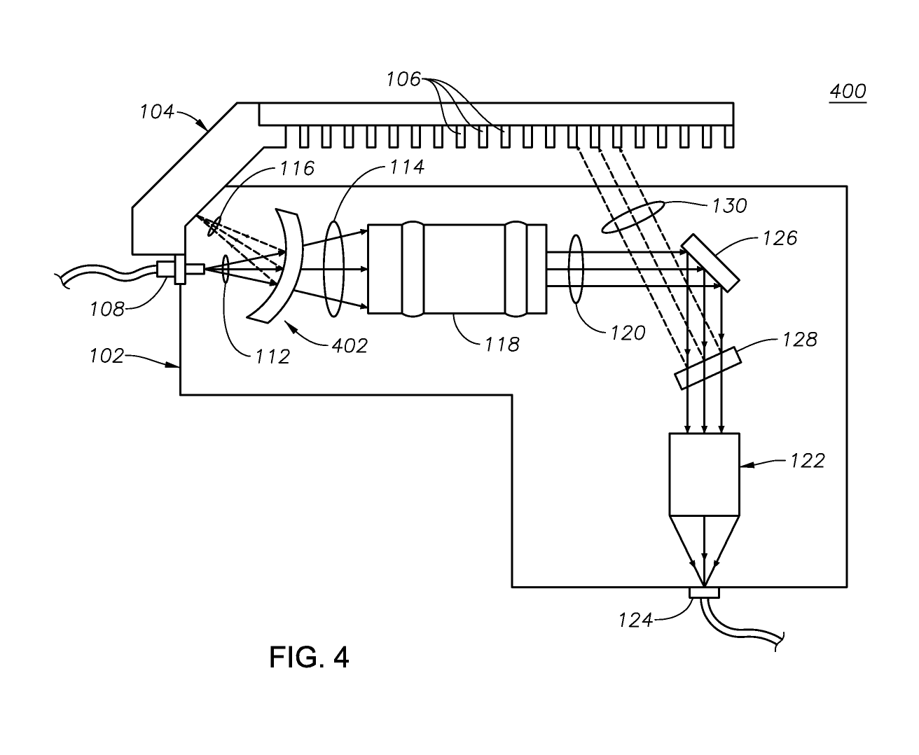

FIG. 2 illustrates an implementation of ophthalmic illumination system 100 in an exemplary surgical console 200, according to certain embodiments of the present disclosure. Surgical console 200 may be any suitable console facilitating ophthalmic surgery. For example, surgical console 200 may include any suitable components for facilitating cataract surgery, vitreo-retinal surgery, refractive surgery, or any other suitable ophthalmic surgery. Although only a portion of ophthalmic illumination system 100 is depicted in FIG. 2, the components of ophthalmic illumination system 100 may be substantially the same as described above with regard to FIG. 1 (or, alternatively, the components of alternative system 400, described below with regard to FIG. 4).

In certain embodiments, the above-described components of ophthalmic illumination system may be integrated into the surgical console 200, and the consumable optical fiber 124 may be coupled to the surgical console via a connection port 202 of the surgical console 200. For example, a consumable optical fiber 124 may be integrated into a fiber connector 204 that is configure to engage the fiber port 202 of surgical console 200 such that alignment between the consumable optical fiber and the condensed light beam exiting condenser 122 is maintained. Accordingly, consumable optical fibers connected to various surgical instruments (e.g., vitrectomy probes, trocar cannulas, or any other suitable surgical instruments) may be selectively attached to surgical console 200 such that lighting may be provided according to surgical needs.

In certain embodiments, the detachable fiber connector 204 may include a front body 206 and a rear body 208. A channel 210 may be formed through each of the front body 206 and the rear body 208. A ferrule 212 may be provided within the channel 210. The consumable optical fiber 124 may be accommodated in the ferrule 212. The connection port 202 may include a cylindrical recess configured to receive the front body 206 of the fiber connector 204. The cylindrical recess may be formed by a cylindrical wall 214, which may surround a portion of the front body 206 of the fiber connector 204 when the fiber connector 204 is connected to the connection port 202. The fiber connector 204 may include a nut 216 including female thread on its inner surface which may interact with male threads provided on an outer surface of the cylindrical wall 214 of the connection port 202 to secure the fiber connector 204 at the connection port 202. The cylindrical recess of the connection port 202 may include an inner end surface 218. An opening may be formed through the inner end surface 218 through which a condensed light beam from condenser 122 may project to be coupled into the consumable optical fiber 124. When the fiber connector 204 is connected to the connection port 202, a proximal end surface of the ferrule 212 may abut against the inner end surface 218 such that a proximal end surface of the consumable optical fiber 124 is positioned at the opening of the inner end surface 218. A sleeve 220 may be provided to accommodate and to position the ferrule 212, such that the proximal end of the consumable optical fiber 124 is precisely positioned at the opening of the inner end surface 218. The sleeve 220 may be formed with a material that is not easily deformed such that the ferrule 212 and the consumable optical fiber 124 may be positioned precisely at the opening of the inner end surface 218. A spring may be provided in the fiber connector 204 to exert a biasing force on the ferrule 212 to tightly secure the ferrule 212 against the inner end surface 218 of the fiber port 202.

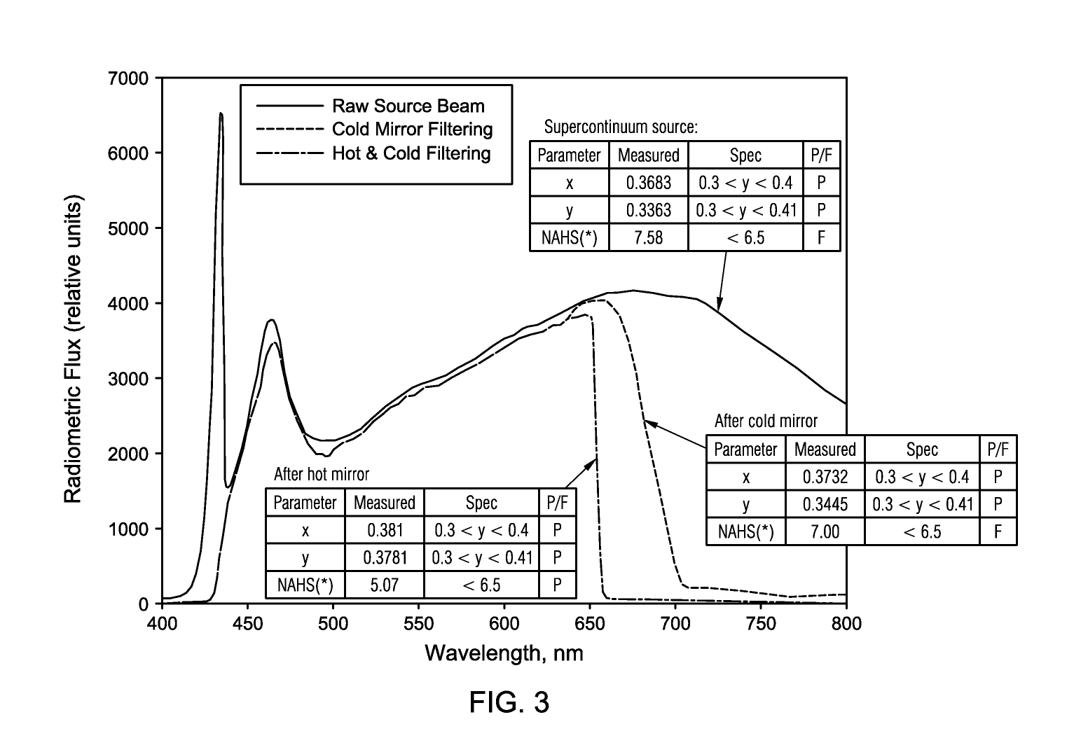

FIG. 3 illustrates a plot of radiometric flux versus wavelength in order to show the filtering properties of ophthalmic illumination system 100. In particular, 3 plots are draw: (1) a plot corresponding to the light beam 110 from light source 108 without filtering by either filtering mirror 112 (cold mirror) or additional filter mirror 126 (hot mirror); (2) a plot corresponding to the light beam 110 from light source 108 with filtering by filtering mirror 112 (cold mirror) but not additional filter mirror 128 (hot mirror); and (3) a plot corresponding to the light beam 110 from light source 108 with filtering by both filtering mirror 112 (cold mirror) and additional filter mirror 128 (hot mirror). As is demonstrated, the filtering of ophthalmic illumination system 100 substantially reduces the amount of IR light emitted by light source 108.

FIG. 4 illustrates an alternative exemplary ophthalmic illumination system 400, according to certain embodiments of the present disclosure. Because certain components of ophthalmic illumination system 400 may be substantially the same as those described above with regard to FIG. 1 (except for relative positioning), like numbering has been use and description has been omitted for the sake of brevity.

The primary differences between ophthalmic illumination system 400 and ophthalmic illumination system 100 depicted in FIG. 1 are that (1) light source 108 in ophthalmic illumination system 400 may be positioned such that its optical axis substantially coincides with that of collimator 118, and (2) filtering mirror 112 (e.g., a flat dichroic cold mirror positioned at an angle relative to light source 108) has been replaced with a filtering mirror 402. In the depicted embodiment, filtering mirror 402 comprises a concave hot mirror coated such that all or a portion of light in the visible spectrum (e.g., IR filtered beam 114) passes through filtering mirror 402 while all or a portion of light in the IR spectrum (IR portion 116) is reflected toward heat sink 104. In certain embodiment, the optical axis of concave filtering mirror 402 may differ from the optical axis of light source 108 such that reflected IR portion 116 does not reflect back into light source 108. Moreover, the radius of curvature of concave filtering mirror 402 may be selected such that reflected IR portion 116 is focused to a particular point or location on heat sink 104. In such embodiments, heat sink 104 may differ from that depicted and may comprise a light pipe into which IR portion 116 may be focused, allowing the light pipe to carry IR portion away from base plate 102 (e.g., to a separate dissipation element).

Although filtering mirror 402 is depicted and described as being a concave hot mirror, the present disclosure contemplates that filtering mirror 402 may include any suitable mirror (e.g., flat, convex, etc.) operable to direct reflected IR light to any suitable heat sink positioned at any suitable location.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by the following claims.

What is claimed is:

1. An ophthalmic illumination apparatus, comprising
a light source operable to generate a light beam;
a filtering mirror configured to generate an infrared (IR) filtered light beam by reflecting at least a portion of wavelengths of the light beam in the visible spectrum while filtering out at least a portion of wavelengths of the light beam in the IR spectrum;
a collimator configured to receive at least a portion of the IR filtered light beam and generate a collimated light beam; and
a condenser configured to couple at least a portion of the collimated light beam into an optical fiber.

2. The ophthalmic illumination apparatus of claim 1, wherein the filtering mirror comprises a dichroic cold mirror.

3. The ophthalmic illumination apparatus of claim 1, wherein axial the light source comprises a supercontinuum source.

4. The ophthalmic illumination apparatus of claim 1, further comprising a heat sink operable to absorb at least a portion of the wavelengths of the light beam in the IR spectrum filtered by the filtering mirror and dissipate heat associated therewith.

5. The ophthalmic illumination apparatus of claim 4, wherein the heat sink is thermally isolated from a base plate to which one or more of the light source, the filtering mirror, the collimator, and the condenser are mounted.

6. The ophthalmic illumination apparatus of claim 4, wherein the heat sink comprises an absorptive region having an antireflective coating to minimize reflection of IR light.

7. The ophthalmic illumination apparatus of claim 4, wherein the heat sink comprises an absorptive region constructed of objects having high aspect ratios to minimize reflection of IR light.

8. The ophthalmic illumination apparatus of claim 1, wherein the collimator comprises a collimator housing including one or more lenses.

9. The ophthalmic illumination apparatus of claim 1, wherein the IR filtered light beam comprises light having wavelengths in the range of 440 nanometers to 660 nanometers.

10. The ophthalmic illumination system of claim 1, further comprising an additional filtering mirror operable to further filter the collimated light beam by reflecting at least a portion of wavelengths of the collimated light beam in the visible spectrum while filtering out at least a portion of wavelengths of the collimated light beam in the IR spectrum.

11. The ophthalmic illumination system of claim 10, wherein the additional filtering mirror comprises a hot mirror.

12. The ophthalmic illumination system of claim 1, wherein the optical fiber comprises an optical fiber terminating at an ophthalmic illumination probe.

13. The ophthalmic illumination system of claim 1, wherein the optical fiber comprises nano fiber having core diameter of 25 μm.

14. An ophthalmic surgical system, comprising
an ophthalmic illumination apparatus, comprising:
a light source operable to generate a light beam;
a filtering mirror configured to generate an infrared (IR) filtered light beam by reflecting at least a portion of wavelengths of the light beam in the visible spectrum while filtering out at least a portion of wavelengths of the light beam in the IR spectrum;
a collimator configured to receive at least a portion of the IR filtered light beam and generate a collimated light beam; and
a condenser configured to focus at least a portion of the collimated light beam; and
a connection port comprising an opening into which the condenser focuses the at least a portion of the collimated light beam, the connection port configured to receive a detachable fiber connector comprising an optical fiber connected to an ophthalmic surgical tool, the at least a portion of the collimated light beam focused by the condenser being transmitted by the optical fiber to the ophthalmic surgical tool.

15. The ophthalmic surgical system of claim 14, wherein the filtering mirror comprises a dichroic cold mirror.

16. The ophthalmic surgical system of claim 14, wherein the ophthalmic surgical tool comprises one of a vitrectomy probe and a trocar cannula.

17. The ophthalmic surgical system of claim 14, further comprising a heat sink operable to absorb at least a portion of the wavelengths of the light beam in the IR spectrum filtered by the filtering mirror and dissipate heat associated therewith.

18. The ophthalmic surgical system of claim 17, wherein the heat sink is thermally isolated from a base plate to which one or more of the light source, the filtering mirror, the collimator, and the condenser are mounted.

19. The ophthalmic surgical system of claim 14, wherein the ophthalmic illumination apparatus further comprising an additional filtering mirror operable to further filter the collimated light beam by reflecting at least a portion of wavelengths of the collimated light beam in the visible spectrum while filtering out at least a portion of wavelengths of the collimated light beam in the IR spectrum.

20. The ophthalmic illumination system of claim 10, wherein the additional filtering mirror comprises a hot mirror.

21. An ophthalmic illumination apparatus, comprising
a light source operable to generate a light beam;
a filtering mirror configured to generate an infrared (IR) filtered light beam by reflecting at least a portion of wavelengths of the light beam in the IR spectrum while letting pass through at least a portion of wavelengths of the light beam in the visible spectrum;
a collimator positioned along the same optical axis as the light source, the collimator configured to receive at least a portion of the IR filtered light beam and generate a collimated light beam; and
a condenser configured to couple at least a portion of the collimated light beam into an optical fiber.

22. The ophthalmic illumination apparatus of claim 21, wherein the filtering mirror comprises a concave hot mirror having an optical axis that differs from the optical axis of the light source.
